(19) United States
(12) Patent Application Publication
(10) Pub. No.: US 2015/0177244 A1
(43) Pub. Date: Jun. 25, 2015

(54) PARALLEL ANALYSIS OF SERUM EPCAM AND MMP7 TO DISCRIMINATE SEPSIS, NECROTIZING ENTEROCOLITIS AND NORMAL CONTROL PATIENTS

(71) Applicant: THE BOARD OF TRUSTEES OF THE LELAND STANFORD JUNIOR UNIVERSITY, Palo Alto, CA (US)

(72) Inventors: Bruce Xuefeng Ling, Palo Alto, CA (US); Karl G. Sylvester, Los Altos, CA (US); Changlin Fu, Palo Alto, CA (US); Shan Xiang Wang, Palo Alto, CA (US); Dokyoon Kim, Stanford, CA (US)

(21) Appl. No.: 14/576,967

(22) Filed: Dec. 19, 2014

Related U.S. Application Data
(60) Provisional application No. 61/919,459, filed on Dec. 20, 2013.

(51) Int. Cl. G01N 33/573; G01N 33/68

(52) U.S. Cl. CPC G01N 33/573; G01N 33/6893; G01N 2333/96494; G01N 2333/4737; G01N 2333/70503; G01N 2800/067; G01N 2800/26

(57) ABSTRACT

Methods and compositions are provided for making a necrotizing enterocolitis (NEC) or sepsis assessment of an individual. Aspects of the methods include detecting a biomarker or panel of biomarkers selected from MMP7, EpCAM, and CRP. In addition, reagents, devices, systems and kits thereof that find use in practicing the subject methods are provided.

### FIELD OF THE INVENTION

This invention pertains to diagnosing Necrotizing Enterocolitis and sepsis.

### BACKGROUND OF THE INVENTION

Necrotizing enterocolitis (NEC) is one of the most common life-threatening diseases of the newborn. NEC predominantly affects low birth weight infants in the first weeks of life with a reported frequency of between 1% and 5% of NICU admissions [1,2] and mortality rates for infants with NEC ranging from 15% to 30%. The pathogenesis of NEC includes progressive inflammation of the gut involving enteric bacteria, the innate immune system, and a compromised intestinal epithelial barrier resulting in eventual necrosis in advanced cases.

Approximately one half of all infants with NEC have mild disease that will recover with medical therapy (medical NEC) [3,4]. This mild form of the disease is very similar to neonatal sepsis. The remaining patients progress to intestinal gangrene with perforation and/or irreversible necrosis requiring emergency surgical intervention (surgical NEC) Several studies have demonstrated that surgical intervention for NEC is an independent risk factor for long-term growth abnormalities, adverse neurodevelopmental outcomes, and gastrointestinal morbidity including short bowel syndrome [3,4]. Improvement in NEC outcomes will require the development of sensitive and specific diagnostic instruments to discriminate NEC from sepsis to enable further study of new medical and surgical therapies as they are developed [5]. This will require NEC-specific biomarkers that can be analyzed in a multiplex format over a broad dynamic range of possible analyte concentrations. An additional improvement upon existing technology would be an assay with a detection limit that exceeds currently available immune-based detection platforms. The present invention addresses these issues.

### SUMMARY OF THE INVENTION

Methods and compositions are provided for making a necrotizing enterocolitis (NEC) or sepsis assessment of an individual. Aspects of the methods include detecting a biomarker or panel of biomarkers selected from MMP7, EpCAM, and CRP. In addition, reagents, devices, systems and kits thereof that find use in practicing the subject methods are provided.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Standard curves measured on magnetic protein chips for human (A) CRP, (B) MMp7, and (C) EpCAM. Red lines are fitting curves, and error bars are ±1 standard deviation.

FIG. 2. Dot plot analysis comparing CRP, MMp7, EpCAM, and MMp7/EpCAM ratio in control, sepsis and NEC subjects.

FIG. 3. ROC analysis of the MMp7/EpCAM ratio as a diagnostic panel discriminating control, sepsis and NEC subjects.

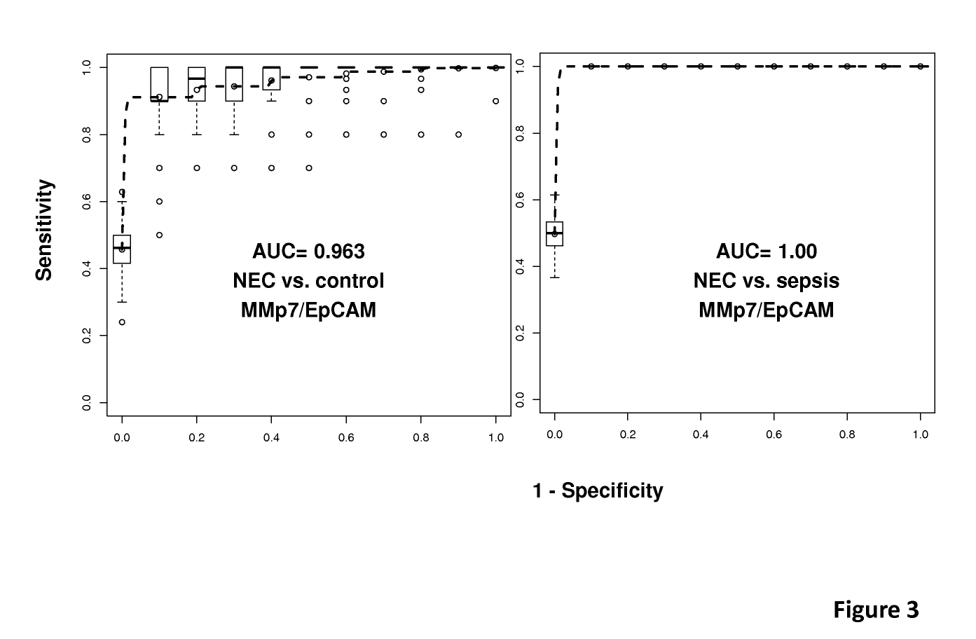

FIG. 4. ROC analysis of the CRP/EpCAM ratio as a diagnostic panel discriminating control, sepsis and NEC subjects.

FIG. 5. ROC analysis of the diagnostic panel of CRP, MMp7 an EpCAM to discriminate control, sepsis and NEC subjects.

### DETAILED DESCRIPTION OF THE INVENTION

Methods and compositions are provided for making a necrotizing enterocolitis (NEC) or sepsis assessment of an individual. Aspects of the methods include detecting a biomarker or panel of biomarkers selected from MMP7, EpCAM, and CRP. In addition, reagents, devices, systems and kits thereof that find use in practicing the subject methods are provided. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, aspects of the subject invention are directed to making a necrotizing enterocolitis (NEC)/sepsis assessment in an individual. By necrotizing enterocolitis, or NEC, it is meant the gastrointestinal condition in which a segment of the intestine becomes necrotic; in some instances, the intestinal region perforates, causing peritonitis and often free intra-abdominal air. Infection and inflammation of the gut are hallmarks of the condition, along with abdominal distention, blood in the stool, diarrhea, feeding intolerance, lethargy, temperature instability, and vomiting. By “sepsis” it is meant a bacterial infection in the context of fever of greater than 38° C. (100.4° F.). Blood pressure drops, resulting in shock. Major organs and systems, including the kidneys, liver, lungs, and central nervous system, stop functioning normally. Infection is typically confirmed by a blood culture that reveals bacteria, blood gases that reveal acidosis, kidney function tests that are abnormal, a platelet count that is lower than normal, and/or a white blood cell count that is lower or higher than normal. Other indications of sepsis include a blood differential that shows immature white blood cells, the presence of higher than normal amounts of fibrin degradation products in the blood, and a peripheral smear that shows a low platelet count and destruction of red blood cells. The treatment is typically antibiotics delivered intravenously. In infants, sepsis may be classified as “early onset” (within the first 7 days of birth), which usually results from organisms acquired intrapartum, and “late onset” (more than 7 days after birth), in which the infection is usually by organisms from the environment.

By making an “NEC/sepsis assessment”, it is meant to include diagnosing NEC or sepsis in an individual, including, for example, discriminating between NEC and sepsis in an individual suspected of having NEC or sepsis; determining a treatment for a patient suspected of having NEC or sepsis, including, for example, diagnosing NEC or sepsis in the individual, and selecting a treatment for the individual based on the diagnosis; and/or monitoring an individual having NEC or sepsis, including, for example, determining the responsiveness of an individual having NEC or sepsis to a therapy. By a “diagnosis” it is generally meant a determination as to whether a subject is presently affected by a disease or disorder, and/or a prognosis of a subject affected by a disease or disorder (e.g., identification of disease states, stages of the disease, likelihood that a patient will die from the disease). By “treatment”, “treating” and the like it is generally meant obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. By “monitoring” it is meant the use of therametrics (e.g., monitoring a subject's condition) to provide information as to the effect or efficacy of therapy.

The terms “individual,” “subject,” “host,” and “patient,” are used interchangeably herein and refer to any mammalian subject for whom diagnosis, treatment, or therapy is desired, particularly humans.

### Biomarkers

In some aspects of the present disclosure, biomarkers are provided for making an NEC/sepsis assessment, e.g. diagnosing NEC or sepsis, monitoring the patient having NEC or sepsis, and/or determining a treatment for a patient suspected of having NEC or sepsis. By a “biomarker”, it is meant molecular entity whose representation in a sample is associated with a clinical phenotype, e.g. NEC or sepsis. For example, a biomarker may be differentially represented, i.e. represented at a different level, in a sample from an individual that will develop or has developed NEC or sepsis as compared to a healthy individual. By differentially represented, it is meant 1.5-fold, 2-fold, 2.5-fold, 3-fold, 4-fold, 5-fold, 7.5-fold, 10-fold, or greater difference—i.e. increase or decrease—in the representation of the biomarker in the sample associated with the clinical phenotype than in a sample not associated with the clinical phenotype.

The subject biomarkers include gene products that are differentially represented in individuals having NEC and/or sepsis as compared to healthy individuals. As used herein, a gene product includes, for example, an unspliced RNA, an mRNA, a splice variant mRNA, a microRNA, a fragmented RNA, a polypeptide, a post-translationally modified polypeptide, a splice variant polypeptide, a peptide, etc. As demonstrated in the examples of the present disclosure, biomarkers of interest to the subject compositions and methods include MMP7 (also known as matrix metallopeptidase 7, matrix metalloproteinase 7, matrilysin, matrin or uterine metalloproteinase), EpCAM (also known as EpiCAM, epithelial cell adhesion molecule, adenocarcinoma-associated antigen, Trop-1, human epithelial glycoprotein-2, GA733-2, or tumor-associated calcium signal transducer 1), and CRP (also known as C-reactive protein, or pentraxin).

In some aspects of the invention, a panel of biomarkers is provided. By a “panel” of biomarkers, it is meant two or more biomarkers, e.g. 3 or more biomarkers, 4 or more biomarkers etc., whose levels, when considered in combination, find use in providing an NEC/sepsis assessment, e.g. diagnosing NEC or sepsis, monitoring the patient having NEC or sepsis, and/or determining a treatment for a patient having NEC or sepsis. In some embodiments, the biomarker panel comprises MMP7 and EpCAM. In some embodiments, the biomarker panel comprises MMP7 and CRP. In some embodiments, the biomarker panel comprises EpCAM and CRP. In some embodiments, the biomarker panel comprises MMP7, EpCAM and CRP. As will be understood by the ordinarily skilled artisan, other biomarkers known in the art may also be included in the subject biomarker panels. See, for example, the biomarkers disclosed in U.S. application Ser. No. 14/094,509, the full disclosure of which is incorporated herein by reference.

### Methods

In practicing methods of the invention, a biomarker signature is obtained for an individual. By a “biomarker signature” it is meant a representation of the level of one or more of the subject biomarkers in a sample, in some instances two or three of the subject biomarkers, i.e. a biomarker panel, and comprises the quantitative data on the levels of these one or more biomarkers in a sample. Examples of biomarker signatures include biomarker profiles, e.g. RNA profiles and protein profiles, and biomarker scores, e.g. RNA scores and protein scores. By a “biomarker profile” it is meant the normalized expression level of one or more genes of interest, more usually two or more genes of interest, in a patient sample. By a “biomarker score” it is meant a single metric value that represents the sum of the weighted expression levels of two or more biomarkers of interest in a patient sample. Weighted biomarker levels are calculated by multiplying the normalized expression level (e.g. as determined by measuring RNA or polypeptide levels) of each gene by its “weight”, the weight of each gene being determined by analysis of a reference dataset, or “training set”, e.g. the datasets provided in the examples section below, e.g. by Principle Component Analysis (PCA), Linear discriminant analysis (LDA), Fisher's linear discriminant analysis, and the like, as are known in the art. Thus, for example, when PCA is used, the expression score is the weighted sum of expression levels of the genes of interest in a sample, where the weights are defined by their first principal component as defined by a reference dataset.

To obtain a biomarker signature, the level of the one or more of the subject biomarkers is measured in a sample from an individual, i.e. the levels of 1 or more, 2 or more, or all three biomarkers. In some instances, the level of one or more additional biomarkers, e.g. as known in the art, is also measured. See, for example, the biomarkers disclosed in U.S. application Ser. No. 14/094,509.

In some embodiments, the individual may appear healthy, i.e. the individual does not have symptoms of NEC or sepsis. In some embodiments, the individual is at risk for having NEC or sepsis. A patient that is at risk for having NEC or sepsis is one in which historical factors, physical findings and/or radiological findings indicate risk for NEC or sepsis. Historical factors include, for example, feeding intolerance (defined as vomiting two or more feedings within 24 hours or any vomit containing bile, or the presence of gastric residuals of volume greater than 6 cc/kg or any aspirate containing bile), apneic/bradycardic episodes, oxygen desaturation episodes, guaiac positive, or bloody stools. Physical findings include, for example, abdominal distention, capillary refill time >2 sec, abdominal wall discoloration, or abdominal tenderness. Radiological findings include, for example, pneumatosis intestinalis, portal venous gas, Ileus, dilated bowel, pneumoperitoneum, air/fluid levels, thickened bowel walls, ascites or peritoneal fluid, or free intraperitoneal air, absent bowel sounds, hypotension, abdominal cellulitis, and right lower quadrant mass.

The level of the subject biomarker(s) may be assessed by any convenient method for measuring a gene product in a sample. For example, an RNA transcript can be detected. The term “RNA transcript” as used herein refers to the RNA transcription products of a gene, including, for example, mRNA, an unspliced RNA, a splice variant mRNA, a microRNA, and a fragmented RNA. As another example, the level of polypeptide may be measured. The term “polypeptide” as used herein and as it is applied to a gene refers to the amino acid product encoded by a gene, including, for example, full length gene product, splice variants of the full length gene product, and fragments of the gene product, e.g. peptides.

The level of the subject biomarker(s) is typically measured by analyzing a body fluid sample, e.g. a sample of urine, blood, or saliva, obtained from an individual. Usually, the sample is a blood sample, e.g. a plasma sample, collected from the individual. The sample that is collected may be freshly assayed or it may be stored and assayed at a later time. If the latter, the sample may be stored by any convenient means that will preserve the sample so that gene expression may be assayed at a later date. For example the sample may be freshly cryopreserved, that is, cryopreserved without impregnation with fixative, e.g. at 4° C., at −20° C., at −60° C., at −80° C., or under liquid nitrogen. Alternatively, the sample may be fixed and preserved, e.g. at room temperature, at 4° C., at −20° C., at −60° C., at −80° C., or under liquid nitrogen, using any of a number of fixatives known in the art, e.g. alcohol, methanol, acetone, formalin, paraformaldehyde, etc.

The sample may be assayed as a whole sample, e.g. in crude form. Alternatively, the sample may be fractionated prior to analysis, e.g. for a blood sample, to purify leukocytes if, e.g., the biomarker to be assayed is RNA or intracellular protein, or to purify plasma or serum if, e.g., the biomarker is a secreted polypeptide. Further fractionation may also be performed, e.g., for a purified leukocyte sample, fractionation by e.g. panning, magnetic bead sorting, or fluorescence activated cell sorting (FACS) may be performed to enrich for particular types of cells, thereby arriving at an enriched population of that cell type for analysis; or, e.g., for a plasma or serum sample, fractionation based upon size, charge, mass, or other physical characteristic may be performed to purify particular secreted polypeptides, e.g. under denaturing or non-denaturing (“native”) conditions, depending on whether or not a non-denatured form is required for detection. One or more fractions are then assayed to measure the expression levels of the one or more genes of interest. The level of the one or more biomarkers of interest may be measured by measuring protein levels, i.e. peptide or polypeptide, levels or by measuring RNA levels.

For measuring protein levels, the amount or level in the sample of one or more biomarker proteins/polypeptides or peptide fragments thereof is determined. In such cases, any convenient protocol for evaluating protein or peptide levels may be employed wherein the level of one or more proteins or peptides in the assayed sample is determined.

While a variety of different manners of assaying for biomarker levels are known in the art, one representative and convenient type of protocol for assaying levels of protein or RNA is a magnetic nanoparticle (MNP) sensor. In an MNP-based detection platform, a proximity label is created by conjugating a magnetic label, e.g. magnetizable nanoparticles, e.g., to an affinity reagent, e.g. antibody or oligonucleotide, which is specific for the biomarker of interest. The magnetizable proximity label is then incubated with the sample under conditions that promote the binding of the proximity label to biomarker protein or RNA (the “analyte”). In an alternative configuration, the magnetizable proximity label is specific for a capture probe, e.g. an antibody, an oligonucleotide, etc. that is specific for the analyte of interest; the capture probe is incubated with the sample under conditions that promote the binding of the capture probe to analyte; and the magnetizable proximity label is incubated with the sample to promote the binding of magnetizable proximity label to capture probe.

The sample comprising analyte bound to magnetizable proximity label is then contacted to the surface of the MNP sensor, which has been functionalized to bind to the analyte. For example, the surface of the MNP sensor may be functionalized to provide for covalent binding or non-covalent association of the analyte and proximity sensor, including, but not limited to, non-specific adsorption, binding based on electrostatic (e.g., ion-ion pair interactions), hydrophobic interactions, hydrogen bonding interactions, and the like. As another example, the surface of the MNP sensor may be functionalized to comprise surface capture ligand(s) that specifically binds to the analyte(s), e.g. antibodies, receptors, oligonucleotides, etc. specific for the biomarker of interest.

Contacting the MNP sensor with an assay composition that includes the analyte of interest bound to magnetizable proximity label results in binding of the analyte to the surface of the proximity sensor. The presence of a magnetic label near the surface of a magnetic proximity sensor, e.g. 200 nm or less, such as 150 nm or less, including 100 nm or less from the sensor surface, induces a detectable change in the magnetic proximity sensor, such as, but not limited to, a change in resistance, conductance, inductance, impedance, etc.

MNP-based multiplex protein detection platforms are able to detect a number of biomolecules in diverse clinical samples (for example, serum, urine, cell lysates or saliva) with high sensitivity (down to attomolar resolution) and large linear dynamic range (more than four decades). In some instances, the methods are wash-free methods of determining the presence of one or more analytes in a sample, i.e. no washing step is performed following reagent and/or sample contact with a sensor surface. As such, no step is performed during the assays of these embodiments in which unbound reagent or unbound sample is removed from the sensor surface. The multianalyte ability, sensitivity, scalability, and ease of use of the MNP-based protein assay technology make it a strong candidate platform for versatile molecular diagnostics in both research and clinical settings. In addition, MNP-based technology can provide a quantitative determination, including both a semi-quantitative determination in which a rough scale result, e.g., low, medium, high, is provided to a user regarding the amount of analyte in the sample, and fine scale results in which an exact measurement of the concentration of the analyte is provided to the user. See, for example, U.S. Pat. No. 7,906,345; PCT Application No. US2008/077111; US Application Publication No. 2011/0027901; and US Application Publication No. 2011/0223612, the full disclosures of which are incorporated herein by reference.

Another well-developed method in the art for measuring protein levels is by ELISA. In ELISA and ELISA-based assays, one or more antibodies specific for the protein biomarkers of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Another well-developed method in the art for measuring protein levels or peptide levels is mass spectrometry (MS). In MS-based methods, a sample (which may be solid, liquid, or gas) is ionized; the ions are separated according to their mass-to-charge ratio, e.g. by magnetic sector, by radio frequencies (RF) quadrupole field, by time of flight (TOF), etc.; the ions are dynamically detected by a mechanism capable of detecting energetic charged particles, and the signal is processed into the spectra of the masses of the particles of that sample. In some instances, tandem mass spectrometry (MS/MS or MS²) may be employed, for example, to determine the sequences of peptides separated by MS. For example, a first mass analyzer isolates one peptide from many entering a mass spectrometer. A second mass analyzer then stabilizes the peptide ions and promotes their fragmentation, e.g. by collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD), surface-induced dissociation (SID), etc. A third mass analyzer then sorts the fragments produced from the peptides. For example, a sample, e.g. a urine sample of the present disclosure, may be applied to an LTQ ion trap mass spectrometer equipped with a Fortis tip mounted nano-electrospray ion source, and the fraction scanned with a mass range of 400-2000 m/z. This first MS scan is followed by two data-dependent scans of the two most abundant ions observed in the first full MS scan. Tandem MS can also be done in a single mass analyzer over time, as in a quadrupole ion trap. In some instances, MS is combined with other technologies, e.g. multiple reaction monitoring (MRM) is coupled with stable isotope dilution (SAD) mass spectrometry (MS), which allowed quantitative assays for peptides to be performed with minimum restrictions and the ease of assembling multiple peptide detections in a single measurement. Other methods for detecting peptides in a sample by MS and measuring the abundance of peptides in a sample are well known in the art; see, e.g. the teachings in US 2010/0163721, the full disclosure of which is incorporated herein by reference.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

As another example, flow cytometry may be employed. In flow cytometry-based methods, the quantitative level of polypeptide or peptide fragment of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

Other representative examples include but are not limited to mass spectrometry, proteomic arrays, xMAP™ microsphere technology, western blotting, and immunohistochemistry.

For measuring mRNA levels, any convenient method for measuring mRNA levels in a sample may be used, e.g. hybridization-based methods, e.g. northern blotting and in situ hybridization (Parker & Barnes, Methods in Molecular Biology 106:247-283 (1999)), RNAse protection assays (Hod, Biotechniques 13:852-854 (1992)), and PCR-based methods (e.g. reverse transcription PCR (RT-PCR) (Weis et al., Trends in Genetics 8:263-264 (1992)). Alternatively, any convenient method for measuring protein levels in a sample may be used, e.g. antibody-based methods, e.g. immunoassays, e.g., enzyme-linked immunosorbent assays (ELISAs), immunohistochemistry, and flow cytometry (FACS). The starting material may be total RNA, i.e. unfractionated RNA, or poly A+RNA isolated from a suspension of cells, e.g. a peripheral blood sample. General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

Examples of methods for measuring mRNA levels may be found in, e.g., the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Additionally or alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

The resultant data provides information regarding the level for each of the biomarkers that have been probed, wherein the information is in terms of whether or not the biomarker is represented in the sample and, typically, at what level, and wherein the data may be both qualitative and quantitative.

Once the level of the one or more biomarkers of interest has been determined, the measurement(s) may be analyzed in any of a number of ways to obtain a biomarker signature.

For example, the biomarker signature may be obtained by analyzing the data to generate a biomarker profile. As used herein, a biomarker profile is the normalized level of one or more biomarkers of interest in a patient sample. A biomarker profile may be generated by any of a number of methods known in the art. For example, the level of each biomarker may be log 2 transformed and normalized relative to the level of a selected housekeeping gene product, e.g. ABL1, GAPDH, or PGK1, or relative to the signal across a whole microarray, etc.

As another example, a biomarker signature may be obtained by analyzed the data to generate a biomarker score. As used herein, a biomarker score is a single metric value that represents the sum of the weighted expression levels of one or more biomarkers of interest in a patient sample. A biomarker score for a patient sample may be calculated by any of a number of methods known in the art for calculating biomarker signatures. For example, the levels of each of the one or more biomarkers of interest in a patient sample may be log 2 transformed and normalized, e.g. as described above for generating a biomarker profile. The normalized levels for each biomarker is then weighted by multiplying the normalized level to a weighting factor, or “weight”, to arrive at weighted expression levels for each of the one or more biomarkers, where the weights are defined by a reference dataset, or “training dataset”, e.g. by Principle Component Analysis, Linear discriminant analysis (LDA), Fishers linear discriminant analysis, etc, of a reference dataset. The weighted expression levels are then totaled and in some cases averaged to arrive at a single weighted expression level for the one or more biomarkers analyzed. In such instances, any dataset relating to patients having NEC or sepsis may be used as a reference dataset. For example, the weights may be determined based upon any of the datasets provided in the examples section below.

This analysis may be readily performed by one of ordinary skill in the art by employing a computer-based system, e.g. using any hardware, software and data storage medium as is known in the art, and employing any algorithms convenient for such analysis.

A biomarker signature so obtained is then employed to provide an NEC/sepsis assessment of an individual. In some embodiments, the making of the NEC/sepsis assessment includes comparing the biomarker signature to a reference biomarker signature, and making the assessment based on the comparison. The terms “reference” and “control” as used herein mean a standardized biomarker signature to be used to interpret the biomarker signature of a given patient and assign a diagnostic, prognostic, and/or responsiveness class thereto. The reference or control is typically a biomarker signature, e.g. biomarker profile or score, that is representative of biomarker levels that are associated with a particular clinical phenotype. In some instances, the comparison will include determining whether a statistically significant match with a biomarker signature of a positive reference, e.g. a reference associated with NEC or sepsis, or a statistically significant difference with a biomarker signature of a negative reference, e.g. a healthy individual, is present, wherein a statistically significant match with a positive reference or a statistically significant difference from a negative reference is indicative of the disease condition in the subject. In some instances, the comparison will include determining whether the biomarker signature for the subject correlates more closely with the positive reference biomarker signature or the negative reference biomarker signature. By “correlates closely”, it is meant is within about 40% of the reference signature, e.g. 40%, 35%, or 30%, in some embodiments within 25%, 20%, or 15%, sometimes within 10%, 8%, 5%, or less, e.g. it is substantially the same as the reference signature.

In some embodiments, the reference biomarker signature is a NEC-positive reference biomarker signature, e.g. the mean or median biomarker signature across a cohort of individuals affected by NEC. In some embodiments, the NEC-positive reference biomarker signature comprises MMP7 levels that are lower than levels in healthy individuals or levels in individuals that have sepsis, EpCAM levels that are greater than levels in healthy individuals or levels in individuals that have sepsis, and/or CRP levels that are greater than levels in healthy individuals or levels in individuals that have sepsis. In certain embodiments in which a NEC-positive reference biomarker signature is employed, the MMP7 levels are about 0 ng/ml-50 ng/ml; the EpCAM levels are about 0.75 ng/ml-6 ng/ml; and/or the CRP levels are about 1.5 ug/ml-10 ug/ml.

In some embodiments, the reference is a sepsis-positive reference biomarker signature, e.g. the mean or median biomarker signature across a cohort of individuals affected by sepsis. In some embodiments, the sepsis-positive reference biomarker signature comprises MMP7 levels that are substantially the same as levels in healthy individuals and higher than levels in individuals having NEC; EpCAM levels that are substantially the same as levels in healthy individuals and lower than levels in individuals that have NEC; and/or CRP levels that are substantially the same as levels in healthy individuals and lower than levels in individuals that have NEC. In certain embodiments in which a sepsis-positive reference biomarker signature is employed, the MMP7 levels are about 60 ng/ml-150 ng/ml, the EpCAM levels in the sepsis-positive reference are about 0 ng/ml-0.75 ng/ml, and or the CRP levels are about 0 ug/ml-1.5 ug/ml.

In some instances, the subject biomarkers and biomarker panels are used in combination with clinical parameters for NEC or sepsis patient stratification, e.g. as known in the art, to provide an NEC or sepsis assessment. In other words, the subject methods for making an NEC or sepsis assessment further comprises detecting one or more clinical parameters associated with NEC or sepsis in the subject, and making the NEC or sepsis assessment based on the biomarker signature and the detected clinical parameters. The assessment of clinical parameters in conjunction with the subject biomarker signature provides an assessment with greater accuracy, specificity and sensitivity.

For example, one common clinically used set of criteria for diagnosing or staging NEC is Modified Bell's criteria, described in detail in Table 1 below.

TABLE 1
Modified Bell's criteria for staging Necrotizing Enterocolitis.

| | | Abdominal | Radiographic | |
|---|---|---|---|---|
| Stage | Systemic signs | signs | signs | Treatment |
| IA | Temperature | Gastric retention, | Normal or | NPO, antibiotics × |
| Suspected | instability, apnea, | abdominal | intestinal | 3 days |
| | bradycardia, | distention, | dilation, mild | |
| | lethargy | emesis, heme- | ileus | |
| | | positive stool | | |
| IB | Same as above | Grossly bloody | Same as above | Same as IA |
| Suspected | | stool | | |
| IIA | Same as above | Same as above, | Intestinal | NPO, antibiotics × |
| Definite, | | plus absent bowel | dilation, ileus, | 7 to 10 days |
| mildly ill | | sounds with or | pneumatosis | |
| | | without | intestinalis | |
| | | abdominal | | |
| | | tenderness | | |
| IIB | Same as above, | Same as above, | Same as IIA, | NPO, antibiotics × |
| Definite, | plus mild | plus absent bowel | plus ascites | 14 days |
| moderately | metabolic | sounds, definite | | |
| ill | acidosis and | tenderness, with | | |
| | thrombocytopenia | or without | | |
| | | abdominal | | |
| | | cellulitis or right | | |
| | | lower quadrant | | |
| | | mass | | |
| IIIA | Same as IIB, | Same as above, | Same as IIA, | NPO, antibiotics × |
| Advanced, | plus hypotension, | plus signs of | plus ascites | 14 days, fluid |
| severely ill, | bradycardia, | peritonitis, | | resuscitation, |
| intact | severe apnea, | marked | | inotropic support, |
| bowel | combined | tenderness, and | | ventilator therapy, |
| | respiratory and | abdominal | | paracentesis |
| | metabolic | distention | | |
| | acidosis, | | | |
| | Disseminated | | | |
| | Intravascular | | | |
| | Coagulation | | | |
| | (DIC), and | | | |
| | neutropenia | | | |
| IIIB | Same as IIIA | Same as IIIA | Same as | Same as IIA, plus |
| Advanced, | | | above, plus | surgery |
| severely ill, | | | pneumo- | |
| perforated | | | peritoneum | |
| bowel | | | | |

“NPO” = nothing by mouth

Other criteria that may be employed include pH value of blood; portal venous gas in x-ray; abdominal ileus in x-ray; the use of a vasopressor prior to diagnosis; abdominal distention; whether cranial ultrasound was done for ivh (intra-ventricular hemorrhage); vasopressor on diagnosis, i.e. the patient is receiving medications that support blood pressure, e.g. inotropes, chronotropes, alpha agonists and the like, e.g. dopamine; ventilation on diagnosis; whether any positive culture of bacteria or fungus was obtained from blood or urine within 5 days of diagnosis; the gestational age of the patient at birth; (and the patient's birth weight.

As another example, the American College of Chest Physicians and the Society of Critical Care Medicine describes several different levels of sepsis (see Table 2, below).

TABLE 2A
Sepsis levels, as described by the American College of Chest Physicians
and the Society of Critical Care Medicine

Sepsis.
Defined as a systemic inflammatory response syndrome (SIRS) in
response to a confirmed infectious process. Infection can be
suspected or proven (by culture, stain, or polymerase chain reaction
(PCR)), or a clinical syndrome pathognomonic for infection. Specific
evidence for infection includes WBCs in normally sterile fluid (such as
urine or cerebrospinal fluid (CSF), evidence of a perforated viscus
(free air on abdominal x-ray or CT scan, signs of acute peritonitis),
abnormal chest x-ray (CXR) consistent with pneumonia (with focal
opacification), or petechiae, purpura, or purpura fulminans
Severe sepsis.
Defined as sepsis with organ dysfunction, hypoperfusion, or hypotension.
Septic shock.
Defined as sepsis with refractory arterial hypotension or hypoperfusion
abnormalities in spite of adequate fluid resuscitation. Signs of systemic
hypoperfusion may be either end-organ dysfunction or serum lactate
greater than 4 mmol/dL. Other signs include oliguria and altered mental
status. Patients are defined as having septic shock if they have sepsis
plus hypotension after aggressive fluid resuscitation (typically upwards
of 6 liters or 40 ml/kg of crystalloid).

TABLE 2B
Symptoms indicating potential sepsis in neonates

Body temperature changes
Breathing problems
Diarrhea
Low blood sugar
Reduced movements
Reduced sucking
Seizures
Slow heart rate
Swollen belly area
Vomiting
Yellow skin and whites of the eyes (jaundice)
A heart rate above 160 can also be an indicator of sepsis, this tachycardia
can present up to 24 hours before the onset of other signs.

TABLE 2C
Clinical parameters for sepsis in neonates.

1. DLC (differential leukocyte count) showing increased numbers of
polymorphs.
2. DLC (differential leukocyte count)having band cells >20%.
3. increased haptoglobins.
4. micro ESR (Erythrocyte Sedimentation Rate) titer >55 mm.
5. gastric aspirate showing >5 polymorphs per high power field.
6. newborn CSF (Cerebrospinal fluid) screen: showing increased cells and
proteins.
7. suggestive history of chorioamnionitis, PROM (Premature rupture of
membranes), etc.

Any clinical parameter(s) known in the art for identifying a patient at risk for developing NEC or sepsis, for diagnosing NEC or sepsis, or for staging NEC or sepsis, may be assessed. In some instances, a clinical signature, representing the aggregate of clinical parameters, may be ascribed to the patient, and the NEC or sepsis assessment made based on the biomarker signature and the clinical signature. In some instances, an aggregate biomarker-clinical signature, representing the aggregate of clinical parameters, is ascribed to the patient, and the NEC or sepsis assessment made based on the aggregate biomarker-clinical signature.

In some embodiments, providing an NEC/sepsis assessment, e.g. a diagnosis of NEC or of sepsis, determining a therapy for a subject having NEC or sepsis, monitoring a subject having NEC or sepsis, etc. includes generating a written report that includes the artisan's assessment of the subject's current state of health i.e. a “diagnosis assessment”, of the subject's prognosis, i.e. a “prognosis assessment”, of possible treatment regimens, i.e. a “treatment assessment” and/or of responsiveness to therapy, i.e. a “prognosis assessment”. Thus, a subject method may further include a step of generating or outputting a report providing the results of a diagnosis assessment, a prognosis assessment, treatment assessment, or a monitoring assessment, which report can be provided in the form of an electronic medium (e.g., an electronic display on a computer monitor), or in the form of a tangible medium (e.g., a report printed on paper or other tangible medium).

### Reagents, Devices and Kits

Also provided are reagents, devices, systems and kits thereof for practicing one or more of the above-described methods. The subject reagents, devices and kits thereof may vary greatly.

Reagents of interest include those mentioned above with respect to the methods of making an NEC or sepsis assessment of an individual. These would include, for example, affinity reagents for detecting the subject biomarker(s), including, for example, antibodies or oligonucleotides specific for MMP7 protein or RNA, respectively; antibodies or oligonucleotides specific for EpCAM protein or RNA, respectively; and/or antibodies or oligonucleotides specific for CRP protein or RNA, respectively; or a cocktail of antibodies or oligonucleotides, e.g. antibodies/oligonucleotides specific for MMP7 and antibodies/oligonucleotides specific for EpCAM; antibodies/oligonucleotides specific for MMP7 and antibodies/oligonucleotides specific for CRP; antibodies/oligonucleotides specific for EpCAM and antibodies/oligonucleotides specific for CRP; and antibodies/oligonucleotides specific for MMP7, antibodies/oligonucleotides specific for EpCAM, antibodies/oligonucleotides specific for CRP. Preferably, the subject affinity reagents bind specifically to the biomarker, e.g. in a magnetic detection system format, in an ELISA format, in an xMAP microsphere format, on a proteomic array, in suspension for analysis by flow cytometry, by western blotting, by dot blotting, by immunohistochemistry, by RNA microarray, or by PCR. Methods for using the same are well understood in the art. These affinity reagents may be provided pre-bound to a solid matrix, for example, the wells of a multi-well dish or the surfaces of xMAP microspheres. Alternatively, these affinity reagents may be provided in solution. Also of interest are reagents that may be used in conjunction with affinity reagents for the detection of biomarkers by one of the aforementioned detection platforms, for example, detectably-labeled secondary antibodies, magnetizable nanoparticles for conjugation to the aforementioned affinity reagent(s), magnetizable nanoparticles conjugated to a ligand that is specific for the aforementioned affinity reagent(s), buffers, dNTPs, and the like.

Also of interest are devices, e.g. devices configured for use in one of the detection formats described above. Such devices would include, for example, a detection element such as a dipstick, a solid matrix, a plate, or an array, e.g., that is functionalized to capture the biomarker(s) of interest, e.g. by comprising one or more of the aforementioned affinity reagents for capturing the biomarker(s) of interest, or by pretreatment to promote non-specific adsorption, binding based on electrostatic (e.g., ion-ion pair interactions), hydrophobic interactions, hydrogen bonding interactions, and the like. In some instances, the device is configured to perform parallel analysis of at least two of the subject biomarkers, e.g. MMP7 and EpCAM, MMP7 and CRP, or EpCAM and CRP. In some instances, the device is configured to perform parallel analysis of all three of the subject biomarkers, i.e. MMP7, EpCAM and CRP. In some instances, the device is configured to detect one or more of the subject biomarkers and one or more additional NEC or sepsis biomarkers as is known in the art. Of particular interest are hand-held devices.

One exemplary device of interest is a magnetic detection biosensor, e.g. a magnetic nanoparticle (MNP)-based biosensor, which is configured to detect protein biomarkers in a fluid sample from a patient. See, for example, U.S. Pat. No. 7,906,345; PCT Application No. US2008/077111; US Application Publication No. 2011/0027901; and US Application Publication No. 2011/0223612, the full disclosures of which are incorporated herein by reference. For example, the biosensor may be functionalized to capture protein. As another example, the biosensor may be functionalized to specifically capture MMP7, EpCAM, and/or CRP. In some instances the magnetic detection biosensor is configured to detect the biomarker in a 1-100 μl sample volume, e.g. a 1-100 μl sample volume, e.g. 1-50 μl, 1-25 μl, 1-10 μl or 1-5 μl sample volume. In some instances, the fluid sample is a blood sample, e.g. a whole blood sample, or a plasma sample.

In some instances, a kit may be provided. As used herein, the term “kit” refers to a collection of reagents and/or devices provided, e.g., sold, together. For example, a kit for detection of the subject biomarkers by a magnetic nanoparticle-based biosensor may include one or more of the following: an MMP7-specific antibody or oligonucleotide; an EpCAM-specific antibody or oligonucleotide; a CRP-specific antibody or oligonucleotide; one or more proximity labels comprising magnetic nanoparticles conjugated to a moiety (e.g. streptavidin) that binds to the MMP7-specific antibody or oligonucleotide (e.g. biotinylated MMP7-specific antibody or oligonucleotide), the EpCAM-specific antibody or oligonucleotide (e.g. biotinylated EpCAM-specific antibody or oligonucleotide), and/or the CRP-specific antibody or oligonucleotide (e.g. biotinylated CRP-specific antibody or oligonucleotide); and a biosensor device.

As another example, a kit for use in the detection of the subject biomarkers by, e.g., flow cytometry, may include one or more of the following: an MMP7-specific antibody or oligonucleotide; an EpCAM-specific antibody or oligonucleotide; a CRP-specific antibody or oligonucleotide; a fluorescently labeled antibody that binds to the MMP7-specific antibody or oligonucleotide, the EpCAM-specific antibody or oligonucleotide, and/or the CRP-specific antibody or oligonucleotide.

As another example, a kit for use in the detection of the subject biomarkers by, e.g., ELISA, may include one or more of the following: an MMP7-specific antibody or oligonucleotide; an EpCAM-specific antibody or oligonucleotide; a CRP-specific antibody or oligonucleotide; a βgal, Alkaline phosphatase, or other colorometrically-labeled secondary antibodies that bind to the MMP7-specific antibody or oligonucleotide, the EpCAM-specific antibody or oligonucleotide, and/or the CRP-specific antibody or oligonucleotide.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

In some instances, a system may be provided. As used herein, the term “system” refers to a collection of reagents and/or devices and/or kits, however compiled, e.g., by purchasing the collection of reagents and devices, or devices and kits, etc. from the same or different sources. Exemplary systems include a system comprising a biosensor, e.g. a magnetic biosensor, an ELISA kit and, xMAP™ microspheres, a proteomic array, a microarray, etc.; a computer system comprising a module configured to determine the biomarker signature for a subject sample; and/or software configured to determine if the individual is suffering from NEC or sepsis or to monitor an individual suffering from NEC or sepsis based on the detected biomarker signature.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Background:

Necrotizing enterocolitis (NEC) is a major source of neonatal morbidity and mortality. There is an ongoing need for a sensitive diagnostic instrument to discriminate NEC from neonatal sepsis. We hypothesized that magnetic nanoparticle-based biosensor analysis of gut injury-associated biomarkers would provide such an instrument.

### Study Design:

We designed a magnetic multiplexed biosensor platform, allowing the parallel plasma analysis of C-reactive protein (CRP), matrix metalloproteinase-7 (MMp7), and epithelial cell adhesion molecule (EpCAM). Neonatal subjects with sepsis (n=5) or NEC (n=10) were compared to control (n=5) subjects to perform a proof of concept pilot study for the diagnosis of NEC using our ultra-sensitive biosensor platform.

### Results:

Our multiplexed NEC magnetic nanoparticle-based biosensor platform was robust, ultrasensitive (Limit of detection LOD: CRP 0.6 pg/ml; MMp7 20 pg/ml; and EpCAM 20 pg/ml), and displayed no cross-reactivity among analyte reporting regents. To gauge the diagnostic performance, bootstrapping procedure (500 runs) was applied: MMp7 and EpCAM collectively differentiated infants with NEC from control infants with ROC AUC of 0.96, and infants with NEC from those with sepsis with ROC AUC of 1.00. The 3-marker panel comprising of EpCAM, MMp7 and CRP had a corresponding ROC AUC of 0.956 and 0.975, respectively.

Conclusion: The exploration of the multiplexed nano-biosensor platform shows promise to deliver an ultrasensitive instrument for the diagnosis of NEC in the clinical setting.

### Methods

Ethics and Sample Collection.

Informed consent was obtained from the parents of all enrolled subjects. This study was approved by the human subjects protection programs at each participating institution (Yale-New Haven Children's Hospital, Lucile Packard Children's Hospital at Stanford University, and the Children's Hospital of Philadelphia). Blood samples were collected and plasma was isolated by centrifuging the collected blood, and stored at −80° C. prior to analysis.

Reagents.

Anti-human CRP antibody (R&D systems, MAB17071), biotinylated anti-human CRP antibody (R&D systems, BAM17072), native human CRP protein (Biospacific, J81600), anti-human MMp7 antibody (R&D systems, MAB9072), biotinylated anti-human MMp7 antibody (R&D systems, BAF2907), recombinant human MMp7 protein (R&D systems, 907-MP-010), anti-human EpCAM antibody (BioMab, EpAb3-5), biotinylated anti-human EpCAM antibody (R&D systems, MAB9601), recombinant human EpCAM protein (R&D systems, 960-EP-050), poly(allylamine hydrochloride) (Polyscience, 71550-12-4), poly(ethylene-alt-maleic anhydride) (Aldrich, 188050), 1× phosphate buffered saline (PBS) (Invitrogen), 1-ethyl-3-(3-dimethylaminopropyl)carbodiimide hydrochloride (EDC) (Thermo scientific), N-hydroxysuccinimide (NHS) (Aldrich), 1% bovine serum albumin (BSA) (Aldrich), biotinylated bovine serum albumin (biotin-BSA) (Pierce), Tween 20 (Aldrich), and streptavidin-coated MicroBeads (Miltenyi, 130-048-101) were used as received and without further purification.

Magnetic Protein Chip Surface Preparation.

The magnetic protein chip was fabricated by previously reported method [22,23]. The chip surface was washed with acetone, methanol, and isopropanol. Subsequently, the surface was further cleaned by exposing to oxygen plasma (Harrick Plasma, PDC-32G) for 3 minutes. Then, the surface was immersed in a 1% aqueous solution of poly(allylamine hydrochloride) for 5 minutes, followed by rinsing with deionized water. The magnetic protein chip was baked at 120° C. for 1 hour. After incubation in a 2% aqueous solution of poly(ethylene-alt-maleic anhydride), the surface was washed again with deionized water and activated by adding a mixture of 1-ethyl-3-[3-dimethylaminopropyl]carbodiimide hydrochloride and N-hydroxysuccinimide in deionized water. A robotic spotter (Scienion, sciFlexarrayer) was then used to deposit capture antibody solution on the magnetic protein chip surface. PBS solutions of anti-human CRP (0.5 mg/ml), anti-human MMp7 (0.5 mg/ml), and anti-human EpCAM (0.5 mg/ml) were deposited on at least 10 sensors on the magnetic protein chip for each solution. Also, 0.1% PBS solutions of BSA and biotin-BSA were placed over 10 sensors as negative and positive controls, respectively. Reference sensors for measurement of electrical background signals were covered with thick silicon oxide to isolate them from surface reactions. Finally, the prepared magnetic protein chip was stored in a humidity chamber at 4° C. before use.

CRP Assay Protocol.

After washing the magnetic protein chip surface with a washing buffer (0.1% BSA and 0.05% Tween 20 in PBS), the surface was blocked with 1% BSA for 1 hour to avoid unwanted adhesion of non-specific biomolecules. Then, the surface was washed again and immersed in a 10000× diluted plasma sample (diluted in dilution buffer, 0.1% BSA and 0.05% Tween 20 in PBS) for 2 hours. The sample solution was washed away using the washing buffer, and a biotinylated anti-human CRP antibody solution with a concentration of 5 μg/ml was added. Following 1 hour incubation with the biotinylated anti-human CRP antibody, the surface was washed again using the washing buffer before measuring CRP signals from the chip. Real-time signals were collected using a custom designed electric read-out system. Briefly, streptavidin-coated magnetic nanoparticles (Miltenyi, streptavidin MicroBeads) were added to the prepared magnetic protein chip to induce an analyte concentration-dependent signal change. The observed signals were converted to corresponding concentrations using standard curves for each biomarker.

MMp7/EpCAM Duplex-Assay Protocol.

Similar procedures as those used in CRP assay were used for the duplex measurement of MMp7 and EpCAM, except that 2× diluted plasma samples and a mixture of biotinylated anti-human MMp7 antibody and biotinylated anti-human EpCAM antibody solutions (final concentration of 5 μg/ml for each antibody) were used.

Statistical Data Analysis.

Patient demographic data was analyzed using the “Epidemiological calculator” (R epicalc package). Student's t test was performed to calculate p values for continuous variables, and Fisher exact test was used for comparative analysis of categorical variables. Hypothesis testing was performed using Student's t-test (two tailed) and Mann-Whitney U-test (two tailed). The biomarker panel score was defined as the ratio between the geometric means of the respective up- and down-regulated protein biomarkers, and was evaluated by ROC curve analysis [24,25]. 500 testing data sets, generated by bootstrapping, from the biosensor data were used to derive estimates of standard errors and confidence intervals for our ROC analysis. The plotted ROC curve is the vertical average of the 500 bootstrapping runs, and the box and whisker plots show the vertical spread around the average.

### Results

Demographics.

In our cohort (Table 1, NEC n=10; sepsis n=5; control n=5), gender, race, gestational age and birth weight, length and head circumference related differences were analyzed between different subject groups. Statistical differences (p-value: <0.05) were observed in birth weight, birth length, and birth head circumference among the three groups. No statistical differences were observed in gender, race and gestation age.

TABLE 1A
Patient Institution information

| Institution | NEC | Sepsis | Control | Total |
|---|---|---|---|---|
| Stanford University | 0 | 5 | 5 | 10 |
| University of Philadelphia | 3 | 0 | 0 | 3 |
| Yale University | 7 | 0 | 0 | 7 |
| Total | 10 | 5 | 5 | 20 |

TABLE 1B
Patient demographics. IQR, interquartile range.

| Charact- | NEC | Sepsis | Control | |
|---|---|---|---|---|
| eristic | (n = 10) | (n = 5) | (n = 5) | p-value |
| Gender | | | | 1 |
| Female | 4 (40%) | 2 (40%) | 2 (40%) | |
| Male | 6 (60%) | 3 (60%) | 3 (60%) | |
| Race | | | | 0.418 |
| Asian | 3 (30%) | 0 (0%) | 2 (40%) | |
| Black | 2 (20%) | 0 (0%) | 0 (0%) | |
| White | 5 (50%) | 5 (100%) | 3 (60%) | |
| Gestation | | | | 0.078 |
| age | | | | |
| (weeks) | | | | |
| median | 26.5 | 26 | 34 | |
| (IQR) | (24.0, 31.0) | (26, 29) | (33, 35) | |
| Birth weight | | | | 0.028* |
| (grams) | | | | |
| median | 810 | 950 | 2560 | |
| (IQR) | (680, 1050) | (710, 1180) | (1900, 2820) | |
| Birth length | | | | 0.042* |
| (cm) | | | | |
| median | 33 | 36.5 | 44 | |
| (IQR) | (32, 35) | (32.0, 37.0) | (42.75, 46.00) | |
| Birth head | | | | 0.042* |
| circum- | | | | |
| ference | | | | |
| (cm) | | | | |
| median | 23.75 | 25.5 | 33 | |
| (IQR) | (22.00, 29.00) | (23.0, 26.5) | (31.250, 33.125) | |

*p-value < 0.05

Magnetic Protein Chip Calibration of Human CRP, MMp7, and EpCAM.

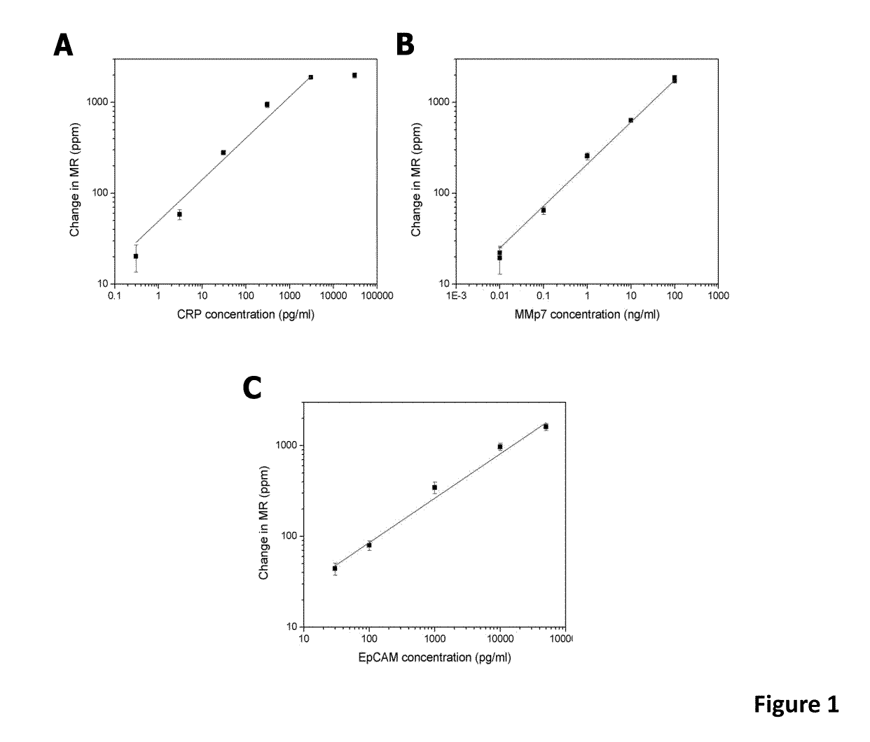

We prepared standard curves to calibrate the magnetic protein chip measurements of CRP, MMp7, and EpCAM in plasma samples (FIG. 1). The standard curves were generated from measurements where each protein was spiked in various concentrations into PBS. The standard dilutions of plasma samples (10,000× for CRP and 2× for MMp7 and EpCAM, respectively) were chosen so that all measured signals were within the linear dynamic range of the standard curve for each protein. We did not observe any cross-reactivity between the reagents used in this study, which was confirmed by the fact that experiments with several mixture combinations of different target proteins and different antibodies did not show any difference in their signals.

The CRP standard curve measured on the magnetic protein chips had a linear dynamic range of more than three orders of magnitude (0.6˜3,000 pg/ml) with an R² value of 0.97 (FIG. 1A). Our magnetic protein chip immunoassay had a detection limit for CRP (0.6 pg/ml) that was lower than currently available commercial ELISA kits (range, generally ˜2 to ˜15 pg/ml) or other multiplex assay platforms such as Luminex and Mesoscale (range, 1.4 to 2 pg/ml for Luminex and 100 pg/ml for Mesoscale). The upper limit of the linear dynamic range for CRP (3,000 pg/ml) was higher than that of ELISA (˜1,000 pg/ml), was similar to that of Luminex (range, 2,000 to 8,000 pg/ml), but was lower than that of Mesoscale (400,000 pg/ml).

The magnetic protein chips showed an MMp7 standard curve covering about four orders of magnitude as its linear dynamic range (10˜100,000 pg/ml) with an R² value of >0.99 (FIG. 1B). The detection limit of MMp7 magnetic protein chip immunoassay was 20 pg/ml, which was lower than that of ELISA (range, generally ˜30 to ˜150 pg/ml), but was higher than that of Luminex (4 pg/ml). The upper limits of the linear dynamic range for MMp7 were similar for magnetic protein chips, ELISA, and Luminex (100 ng/ml for magnetic protein chips, ranges from 2 to 100 ng/ml for ELISA, and 60 ng/ml for Luminex). A Mesoscale kit for MMp7 was not available commercially at the time of this study.

FIG. 1C shows the standard curve for EpCAM measured on the magnetic protein chips. It has a linear dynamic range of more than three orders of magnitude (20˜50,000 pg/ml) with an R² value of 0.96. The detection limit of EpCAM magnetic protein chip immunoassay (20 pg/ml) was lower than that of ELISA (range, generally ˜20 to ˜50 pg/ml), but was comparable to that of Luminex (13.7 pg/ml). The upper limit of the linear dynamic range was highest for the magnetic protein chips (50,000 pg/ml) compared with ELISA (range, ˜6,000 to ˜12,000 pg/ml).

CRP, MMp7, and EpCAM Concentrations in Plasma of NEC, Sepsis, and Healthy Control Infants.

Using the standard curves shown in FIG. 1, we tested the ability of our magnetic protein chip platform to detect the concentration differences of CRP, MMp7, and EpCAM in blood plasma collected from infants with NEC, infants with sepsis, and healthy infants. We performed the immunoassay using two magnetic protein chips per plasma sample, one for CRP assay, and the other for duplex assay of MMp7 and EpCAM.

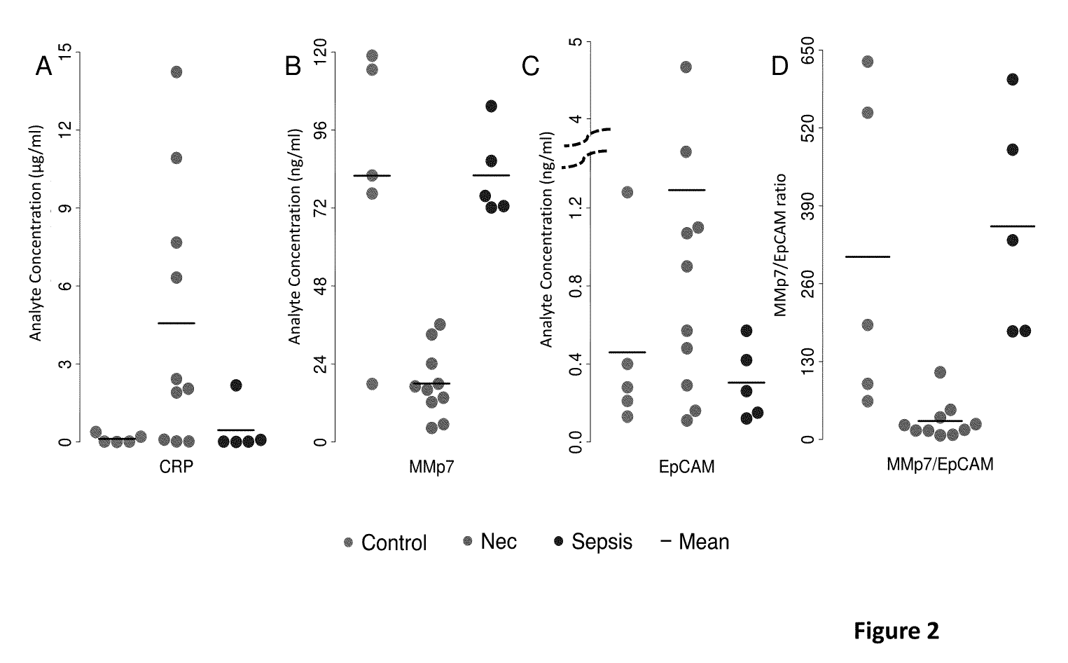

As shown in FIG. 2A, the concentrations of CRP were 4.6±5.0 μg/ml (range, ˜0 to 14.2) in the NEC, 0.5±1.0 μg/ml (range, ˜0 to 2.2) in the sepsis, and 0.1±0.2 μg/ml (range, ˜0 to 0.4) in the healthy control samples. Although the average concentration of CRP was much higher in the NEC samples than in the sepsis or healthy control samples, there were five and three NEC samples whose CRP concentrations were within the CRP concentration range of the sepsis and healthy control samples, respectively. The concentrations of MMp7 (FIG. 2B) were 18.0±10.5 ng/ml (range, ˜4.3 to 36.1) in the NEC, 82.0±13.2 ng/ml (range, ˜72.1 to 103.3) in the sepsis, and 82.0±40.5 ng/ml (range, ˜17.8 to 118.8) in the healthy control samples. The average concentration of MMp7 in the NEC samples was four times less than that of the sepsis or healthy control samples, and the concentration range of the NEC samples was relatively well separated from those of the sepsis and healthy control samples. The concentrations of EpCAM (FIG. 2C) were 1.3±1.6 ng/ml (range, ˜0.1 to 4.7), 0.3±0.2 ng/ml (range, ˜0.1 to 0.6), and 0.5±0.5 ng/ml (range, ˜0.1 to 1.3) in the NEC, sepsis, and healthy control samples, respectively. The higher average concentration of EpCAM observed for the NEC samples was mainly due to two samples which showed about 7-fold higher concentrations than the remainder of the NEC samples. The concentration of EpCAM in the NEC samples after excluding those two samples was 0.6±0.4 ng/ml (range ˜0.1 to 1.1), which is similar to those of sepsis or healthy control samples.

Table 2 lists the p values calculated using Mann-Whitney U test. The CRP concentrations in the NEC samples were significantly different from those of the sepsis or healthy control samples (p<0.05). However, CRP concentration difference between sepsis and healthy control samples was not significant (p=1.0000). MMp7 also showed a significant concentration difference between NEC samples and sepsis samples, and between NEC samples and healthy control samples (p<0.05). Again, however, MMp7 concentration difference between sepsis and healthy control samples was not significant (p=0.4647). EpCAM did not show significant concentration difference (p value >0.05) among the sample groups.

TABLE 2
Comparative analysis of the plasma abundance
between disease categories.

| Analyte | Nec vs. Control | Nec vs. Sepsis | Sepsis vs. Control |
|---|---|---|---|
| CRP (pg/ml) | 0.023218* | 0.031782* | 1.00000 |
| MMp7 (ng/ml) | 0.007992 | 0.000666** | 0.547619 |
| EpCAM (ng/ml) | 0.309603 | 0.111027 | 0.841270 |

*p < 0.05;
**p < 0.005

A Panel of MMp7 and EpCAM to Discriminate NEC and Sepsis.

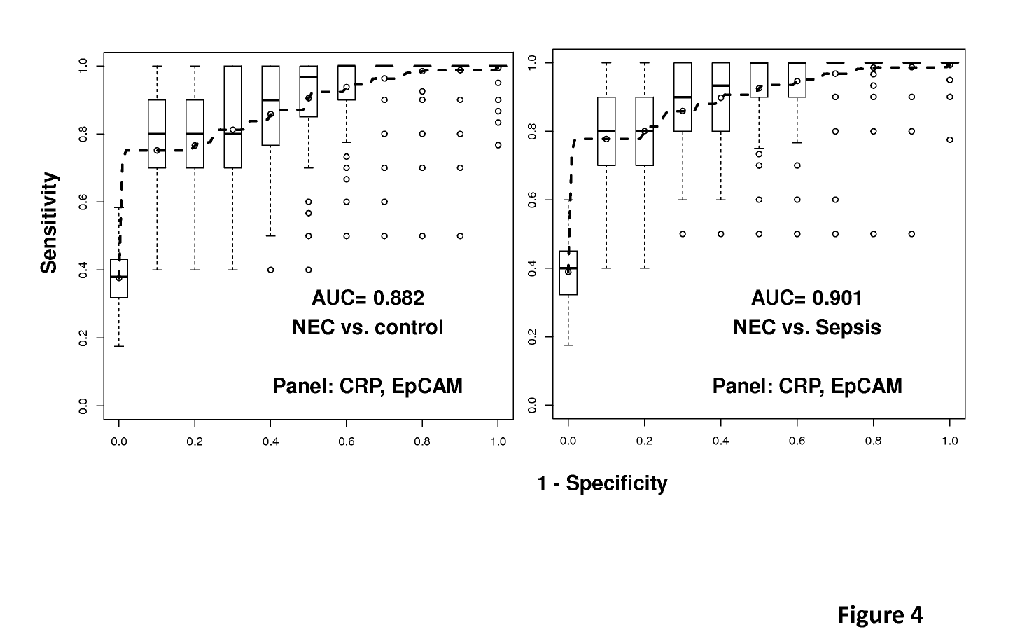

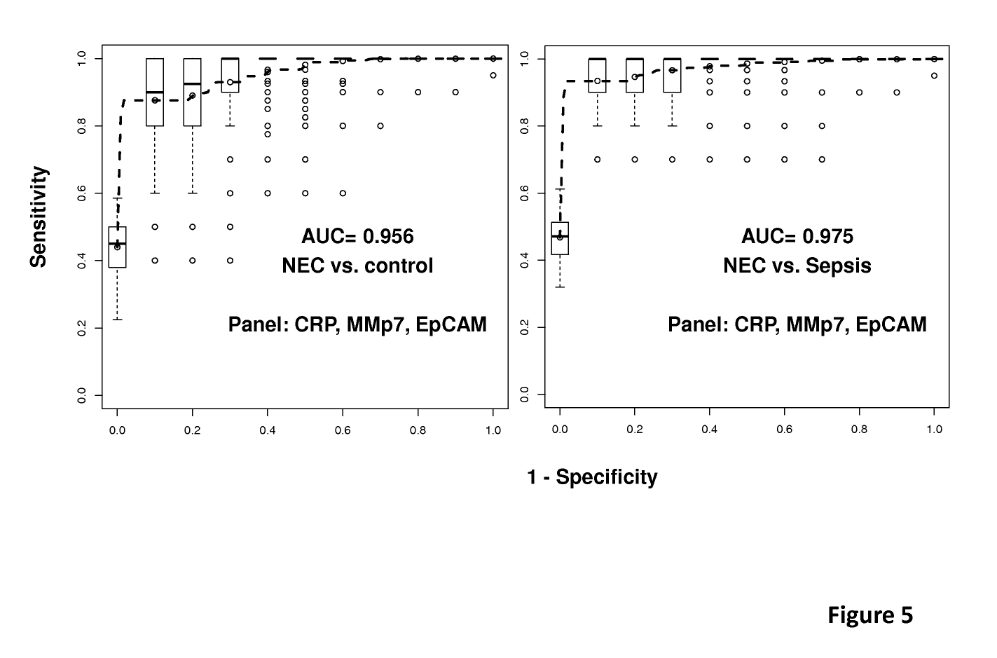

Using MNP biosensor data, we constructed a panel of MMp7 and EpCAM, and the ratio of the two analytes was tested in assessing NEC and sepsis (FIG. 2D). The MMp7/EpCAM ratio's NEC and sepsis discriminant utility was demonstrated in this study (NEC vs. control, ROC AUC 0.963; NEC vs. sepsis, ROC AUC 1.00). Other possible panel constructions, including CRP/EpCAM raito (FIG. 4, NEC vs. control, ROC AUC 0.882; NEC vs. sepsis, ROC AUC 0.901) or combining CRP, MMp7 and EpCAM (FIG. 5, NEC vs. control, ROC AUC 0.956; NEC vs. sepsis, ROC AUC 0.975), were also evaluated. However, MMp7/EpCAM ratio was demonstrated to be the best panel in regard to the discrimination of NEC, sepsis and control subjects.

### Discussion

The clinical presentation of NEC is very similar to that of neonatal sepsis and there are no reliable diagnostic instruments to aid in discriminating these conditions. Clinicians have therefore utilized combinations of non-specific clinical and laboratory indicators to guide patient management. NEC is ultimately diagnosed through a combination of clinical, radiographic, and laboratory findings that in aggregate define the original diagnostic Bell's criteria. This study tested the hypothesis that gut injury and remodeling associated proteins (CRP, MMp7, and EpCAM) could be multiplexed on an ultrasensitive and matrix insensitive biosensor platform to aid in the diagnosis of NEC. ROC curve analysis demonstrated that the MMp7/EpCAM ratio maintains robust performance characteristics. This is encouraging and further suggests an additional advantage of this type of ultrasensitive biosensor platform, which has the capacity to stratify low concentration biomarkers for categorical diagnostic discrimination and may allow early disease state detection. Thus, the integration of the aforementioned biomarkers and diagnostic biosensor platforms into clinical practice will facilitate iterative patient sample testing to guide treatment strategies throughout the course of disease progression and recovery.

### REFERENCES

1. Guthrie S O, Gordon P V, Thomas V, Thorp J A, Peabody J, et al. (2003) Necrotizing enterocolitis among neonates in the United States. J Perinatol 23: 278-285.
2. Kamitsuka M D, Horton M K, Williams M A (2000) The incidence of necrotizing enterocolitis after introducing standardized feeding schedules for infants between 1250 and 2500 grams and less than 35 weeks of gestation. Pediatrics 105: 379-384.
3. Blakely M L, Tyson J E, Lally K P, McDonald S, Stoll B J, et al. (2006) Laparotomy versus peritoneal drainage for necrotizing enterocolitis or isolated intestinal perforation in extremely low birth weight infants: outcomes through 18 months adjusted age. Pediatrics 117: e680-687.
4. Hintz S R, Kendrick D E, Stoll B J, Vohr B R, Fanaroff A A, et al. (2005) Neurodevelopmental and growth outcomes of extremely low birth weight infants after necrotizing enterocolitis. Pediatrics 115: 696-703.
5. Lin P W, Stoll B J (2006) Necrotising enterocolitis. Lancet 368: 1271-1283.
6. Pourcyrous M, Korones S B, Yang W, Boulden T F, Bada H S (2005) C-reactive protein in the diagnosis, management, and prognosis of neonatal necrotizing enterocolitis. Pediatrics 116: 1064-1069.
7. Cetinkaya M, Ozkan H, Koksal N, Akaci O, Ozgur T (2011) Comparison of the efficacy of serum amyloid A, C-reactive protein, and procalcitonin in the diagnosis and follow-up of necrotizing enterocolitis in premature infants. J Pediatr Surg 46: 1482-1489.
8. Povoa P, Almeida E, Moreira P, Fernandes A, Mealha R, et al. (1998) C-reactive protein as an indicator of sepsis. Intensive Care Med 24: 1052-1056.
9. Derikx J P, Evennett N J, Degraeuwe P L, Mulder T L, van Bijnen A A, et al. (2007) Urine based detection of intestinal mucosal cell damage in neonates with suspected necrotising enterocolitis. Gut 56: 1473-1475.
10. Guthmann F, Borchers T, Wolfrum C, Wustrack T, Bartholomaus S, et al. (2002) Plasma concentration of intestinal- and liver-FABP in neonates suffering from necrotizing enterocolitis and in healthy preterm neonates. Mol Cell Biochem 239: 227-234.
11. Sylvester K G, Ling X B, Liu G Y, Kastenberg Z J, Ji J, et al. (In Press) A novel urine peptide biomarker-based algorithm for the prognosis of necrotising enterocolitis in human infants. Gut.
12. Sylvester K G, Ling X B, Liu G Y, Kastenberg Z J, Ji J, et al. (In Press) Urine protein biomarkers for the diagnosis and prognosis of necrotizing enterocolitis in human infants. Gut.
13. Thuijls G, Derikx J P, van Wijck K, Zimmermann L J, Degraeuwe P L, et al. (2010) Non-invasive markers for early diagnosis and determination of the severity of necrotizing enterocolitis. Ann Surg 251: 1174-1180.
14. Rabinowitz S S, Dzakpasu P, Piecuch S, Leblanc P, Valencia G, et al. (2001) Platelet-activating factor in infants at risk for necrotizing enterocolitis. J Pediatr 138: 81-86.
15. Young C, Sharma R, Handfield M, Mai V, Neu J (2009) Biomarkers for infants at risk for necrotizing enterocolitis: clues to prevention? Pediatr Res 65: 91R-97R.
16. Chaaban H, Shin M, Sirya E, Lim Y P, Caplan M, et al. (2010) Inter-alpha inhibitor protein level in neonates predicts necrotizing enterocolitis. J Pediatr 157: 757-761.
17. Evennett N J, Hall N J, Pierro A, Eaton S (2010) Urinary intestinal fatty acid-binding protein concentration predicts extent of disease in necrotizing enterocolitis. J Pediatr Surg 45: 735-740.
18. Pourcyrous M, Bada H S, Korones S B, Baselski V, Wong S P (1993) Significance of serial C-reactive protein responses in neonatal infection and other disorders. Pediatrics 92: 431-435.
19. Bister V, Salmela M T, Heikkila P, Anttila A, Rintala R, et al. (2005) Matrilysins-1 and -2 (MMP-7 and -26) and metalloelastase (MMP-12), unlike MMP-19, are up-regulated in necrotizing enterocolitis. J Pediatr Gastroenterol Nutr 40: 60-66.
20. Trzpis M, Popa E R, McLaughlin P M, van Goor H, Timmer A, et al. (2007) Spatial and temporal expression patterns of the epithelial cell adhesion molecule (EpCAM/EGP-2) in developing and adult kidneys. Nephron Exp Nephrol 107: e119-131.
21. Trzpis M, McLaughlin P M, de Leij L M, Harmsen M C (2007) Epithelial cell adhesion molecule: more than a carcinoma marker and adhesion molecule. Am J Pathol 171: 386-395.
22. Gaster R S, Hall D A, Nielsen C H, Osterfeld S J, Yu H, et al. (2009) Matrix-insensitive protein assays push the limits of biosensors in medicine. Nat Med 15: 1327-1332.
23. Osterfeld S J, Yu H, Gaster R S, Caramuta S, Xu L, et al. (2008) Multiplex protein assays based on real-time magnetic nanotag sensing. Proc Natl Acad Sci USA 105: 20637-20640.
24. Zweig M H, Campbell G (1993) Receiver-operating characteristic (ROC) plots: a fundamental evaluation tool in clinical medicine. Clin Chem 39: 561-577.
25. Sing T, Sander O, Beerenwinkel N, Lengauer T (2005) ROCR: visualizing classifier performance in R. Bioinformatics 21: 3940-3941.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of making an NEC/sepsis assessment of a subject, the method comprising:
detecting the level of MMP7 in a blood sample from a subject to arrive at a biomarker signature representative of the level of MMP7 in the sample, and
making the NEC/sepsis assessment of a subject based upon the biomarker signature.

2. The method according to claim 1, further comprising detecting the level of EpCAM in the blood sample to arrive at a biomarkers signature representative of the levels of MMP7 and EpCAM in the sample, and
making the NEC/sepsis assessment based upon the biomarker signature.

3. The method according to claim 2, further comprising detecting the level of C-reactive protein (CRP) in the blood sample to arrive at a biomarkers signature representative of the levels of MMP7, EpCAM, and CRP in the sample, and
making the NEC/sepsis assessment based upon the biomarker signature.

4. The method according to claim 1, wherein the assessment is a diagnosis of the subject, and the subject is at risk for having NEC or sepsis.

5. The method according to claim 1, wherein the assessment is a monitoring of the subject, and the subject has NEC or sepsis.

6. The method according to claim 1, wherein the assessment is a determination of treatment, and the method comprises selecting a treatment for the subject based upon the NEC/sepsis assessment.

7. The method according to claim 1, wherein the detecting comprises the use of a magnetic nanoparticle sensor.

8. The method according to claim 1, wherein the making of the NEC/sepsis assessment comprises comparing the biomarker signature to a reference biomarker signature, and making the assessment based on the comparison.

9. The method according to claim 8, wherein the reference biomarker signature is an NEC-positive reference biomarker signature and/or a sepsis-positive reference biomarker signature.

10. (canceled)

11. A device for making an NEC or sepsis assessment, the device comprising a magnetic nanoparticle sensor configured to detect one or more proteins selected from MMP7, EpCAM and CRP in a fluid sample from a subject.

12. The device according to claim 11, wherein the magnetic nanoparticle sensor is configured to detect MMP7 and EpCAM.

13. The device according to claim 11, wherein the magnetic nanoparticle sensor is configured to detect MMP7, EpCAM, and CRP.

14. The device according to claim 11, wherein the fluid sample is a blood sample.

15. The device according to claim 11, wherein the fluid sample is a plasma sample.

16. The device according to claim 14, wherein the device is configured to detect protein in a 1-100 ul sample volume.

17. A system for diagnosing NEC or sepsis, the system comprising a device according to claim 11 and one or more references selected from a NEC-positive reference and a sepsis-positive reference.

18. A kit comprising:
one or more biotinylated antibodies selected from a biotinylated antibody specific for MMP7, a biotinylated antibody specific for EpCAM, and a biotinylated antibody specific for CRP; and
streptavidin-labeled magnetic nanoparticles.

19. The kit according to claim 18, further comprising:
a device according to claim 11.
